(19) United States
(12) Patent Application Publication
(10) Pub. No.: US 2009/0306750 A1
(43) Pub. Date: Dec. 10, 2009

(54) Lead Fixation Assembly and Methods of Using Same

(75) Inventors: C. Lance Boling, San Jose, CA (US); Dennis Potts, Scotts Valley, CA (US)

Correspondence Address: NEUROPACE, INC., 1375 SHOREBIRD WAY, MOUNTAIN VIEW, CA, 94043, US

(73) Assignee: NeuroPace, Inc., Mountain View, CA (US)

(21) Appl. No.: 12/135,056

(22) Filed: Jun. 6, 2008

(51) Int. Cl. A61N 1/05

(52) U.S. Cl. 607/116

(57) ABSTRACT

A lead fixation assembly and method of using the same are disclosed. The lead fixation assembly includes a cover and a cap. The cover includes an aperture extending through a cover body and is adapted to fit within an opening formed or occurring in a patient's body. One or more leads may be oriented through the aperture before or after fitting the cover in such an opening. The cover body includes a top surface having a plurality of elements. Each lead oriented through the aperture is arranged on or in and among the elements and may be secured thereby. The cap is adapted to be removably secured to the cover, minimize exposure to the patient via the opening, and, optionally, secure the leads. The leads are passed through an opening in the cap to enable the leads to extend away from the body.

### BACKGROUND

1. Technical Field

The disclosed embodiments generally relate to systems and devices that control the movement of leads after they have been implanted at a desired site in a patient's body. More particularly, the disclosed embodiments relate to systems and devices for affixing a lead within a burr hole that is formed in a patient's skull to gain access to the brain.

2. Background

Neurostimulation systems, and increasingly implantable neurostimulation systems, are used to treat various neurological diseases and other neurological disorders, such as epilepsy, movement disorders (e.g., Parkinson's disease) and chronic pain. Research is ongoing concerning use of implantable neurostimulation systems to treat psychological disorders (e.g., depression), headaches and Alzheimer's disease and to facilitate stroke recovery.

A typical neurostimulation system comprises a stimulation source, such as a pulse generator, that provides stimulation to target neural tissue via one or more leads connected to the stimulation source. Each lead has one or more electrodes designed to be placed on a surface of the brain (cortical electrodes) or within the brain (deep brain electrodes). A signal is transmitted from the stimulation source to the electrode(s), and thus to the desired site in the brain. Some systems also have the capacity to detect and respond to signals detected from one or more of the electrodes through the leads (e.g., “responsive neurostimulators” or other “closed-loop” devices).

Access to the desired portion of the brain is commonly achieved by drilling a hole in a patient's skull (cranium). A cranial drill, sometimes referred to as a “burr”, is used to drill the hole through the outer table, cancellous bone, and inner table of the cranium.

A lead with one or more electrodes on its distal end is introduced into the burr hole and manipulated from outside the patient until the electrodes are positioned at the desired location. Leads with cortical strip electrodes are designed to lay on a surface of the brain. The location at which a cortical strip electrode is placed, for example, may correspond to an area of brain tissue which has previously been identified as the likely focus of seizure activity, for example, using magnetic resonance imaging or some other diagnostic or clinical procedure. Leads with deep brain electrodes are designed to be pushed at least partly into the brain tissue, so that the electrodes rest at or near a target structure (e.g., hypothalamus, subthalamic nucleus, etc.).

Maintaining the electrodes at the desired location once the leads have been implanted is often critical to the purpose of the implant (e.g., delivering stimulation therapy, monitoring a sensed brain signal, etc.). Thus, once one or more leads are placed in the desired areas on or in the brain, the proximal portions of the leads (i.e., a portion of each lead that extends away from the implant site and exteriorly of the burr hole) commonly are secured to prevent the electrodes from being inadvertently dislodged from the location at which the distal ends of the leads bearing the electrodes have been placed. One or all of the components that are used to secure the leads at the site of the burr hole commonly are put into place in the burr hole before the leads are implanted.

Typically, the leads are permitted some play or give after they have been secured, to allow for some relative movement of the leads and the brain or skull, for example, during some sort or head trauma. A device used to secure the proximal portions of the leads also often is provided with a feature that allows the hole to be sealed or substantially sealed to minimize infection from outside agents, such as a cap with a slot through which the leads can be extended and then routed to measuring or stimulation components.

Burr hole cover assemblies and other lead fixation devices include those described in U.S. Pat. No. 7,204,840 to Skakoon et al.; U.S. Pat. No. 6,482,182 to Carroll et al.; U.S. Pat. No. 6,321,104 to Gielen et al.; U.S. Pat. No. 6,210,417 to Baudino et al.; U.S. Pat. No. 6,134,477 to Knuteson; U.S. Pat. No. 6,044,304 to Baudino; U.S. Pat. No. 5,961,519 to Bruce et al.; U.S. Pat. No. 5,927,277 to Baudino et al.; U.S. Pat. No. 5,865,842 to Knuth et al.; and U.S. Pat. No. 5,464,446 to Dreessen et al.

### SUMMARY

Before the present systems, devices and methods are described, it is to be understood that this disclosure is not limited to the particular systems, devices and methods described, as these may vary. It is also to be understood that the terminology used in the description is for the purpose of describing the particular versions or embodiments only, and is not intended to limit the scope.

It must also be noted that as used herein and in the appended claims, the singular forms “a,” “an,” and “the” include plural references unless the context clearly dictates otherwise. Thus, for example, reference to a “lead” is a reference to one or more leads and equivalents thereof known to those skilled in the art, and so forth. Unless defined otherwise, all technical and scientific terms used herein have the same meanings as commonly understood by one of ordinary skill in the art. Although any methods, materials, and devices similar or equivalent to those described herein can be used in the practice or testing of embodiments, the preferred methods, materials, and devices are now described. All publications mentioned herein are incorporated by reference. Nothing herein is to be construed as an admission that the embodiments described herein are not entitled to antedate such disclosure by virtue of prior invention.

In an embodiment, a lead fixation assembly includes a cover sized and shaped to be secured within a hole formed in the body of a patient and having a first aperture. A top surface of the cover includes a first plurality of elements configured to receive a portion of a lead. The lead fixation assembly further includes a cap having a second aperture designed to have a lead pass therethrough. The cap is configured to be removably secured to the cover.

In an embodiment, a method of securing a lead in a lead fixation assembly is provided, the lead having a distal portion intended to be implanted in a patient and a proximal portion intended to extend away from the implant site, the lead having at least one electrode at the distal portion thereof, the lead fixation assembly including a cover sized and shaped to be secured within a hole formed or otherwise occurring in a patient and having a first aperture and a plurality of elements adapted to receive the proximal portion of the lead, and a cap having a second aperture and configured to be removably secured to the cover, the method including installing the cover in the hole, placing the lead extending away from the implant site in the first aperture, placing the proximal portion of the lead on or in and among the plurality of elements of the cover, and removably securing the cap to the cover to affix the proximal portion of the lead between the cap and the plurality of elements such that the lead extending away from the implant site passes through the second aperture.

In an embodiment, a lead fixation assembly includes a cover and a cap. The cover has a cover body having a periphery and a top surface and an aperture extending through the cover body to allow passage therethrough of one or more leads. The cover is adapted to fit within a defect or other opening formed or occurring in a body of a patient. The top surface of the cover has a plurality of elements adapted to receive a proximal portion of each lead. The cap is adapted to be juxtaposed with the cover and has an opening configured to allow passage therethrough or one or more leads.

In an embodiment, a method for using a lead fixation assembly includes threading a proximal end of a lead implanted in a body of a human patient through an aperture in a cover having a cover body, a periphery, and a top surface characterized by a plurality of elements configured to receive a proximal portion of the lead, securing the cover in a hole formed or otherwise occurring in a patient, arranging the proximal portion of the lead on or in and among the plurality of elements, and removably securing a cap to the cover. The cap includes an opening configured to pass the lead therethrough.

In an embodiment, a method for using a lead fixation assembly includes securing a cover having an aperture, a cover body, a periphery and a top surface characterized by a plurality of elements configured to receive a proximal portion of a lead in a hole formed or otherwise occurring in a patient, threading a distal end of the lead through the aperture interiorly of the hole, implanting the distal end of the lead in the body of the patient, arranging the proximal portion of the lead on or in and among the plurality of elements, and removably securing a cap to the cover. The cap includes an opening configured to pass the lead therethrough.

In an embodiment, a method for affixing a lead implanted in the body of a patient in a hole formed or otherwise occurring in a patient includes orienting the lead in an aperture in a cover through a gap in the cover and arranging a proximal portion of the lead on or in and among a plurality of elements provided in a top surface of the cover to substantially prevent the proximal portion of the lead from moving relative to the cover.

### BRIEF DESCRIPTION OF THE DRAWINGS

Aspects, features, benefits and advantages of the embodiments described herein will be apparent with regard to the following description, appended claims and accompanying drawings where:

FIGS. 1A-B are top and side views, respectively, of an exemplary cover for use in a lead fixation assembly according to an embodiment.

FIGS. 1C-D are top views of alternate exemplary covers for use in a lead fixation assembly according to an embodiment.

FIG. 2 is a representational view of an exemplary lead fixation assembly cover located in a cranium according to an embodiment.

FIGS. 3A-B are schematic representations from a side perspective of exemplary raised surfaces for use with a lead fixation assembly cover according to embodiments.

FIG. 3C is a schematic representation from a cross-sectional view of a plurality of recesses or indentations for use with a lead fixation assembly cover according to embodiments.

FIG. 4 is a top view of an exemplary cap for use in a lead fixation assembly according to an embodiment.

FIG. 5 is a representational view of a portion of a cover and cap of a lead fixation assembly according to an embodiment.

FIGS. 6A-B are schematic representations of stimulation systems using lead fixation assemblies according to embodiments.

FIG. 7 depicts a top view of an exemplary cover with a lead according to an embodiment.

FIG. 8 is a flow diagram of an exemplary method of affixing a lead in a lead fixation assembly according to an embodiment.

### DETAILED DESCRIPTION

The lead fixation assemblies disclosed herein are designed to secure leads to discourage or minimize movement of lead electrodes after the electrodes have been placed in the interior of the body, especially in the cranium. In an embodiment, the surgeon can leave some play or give in the lead between the brain or other implant site and the lead fixation assembly in order to account for some relative movement of the lead and brain after lead implantation, for example, acute movement as a result of a trauma from a car accident or blow to the head. However, it is intended that once a lead is implanted, the lead fixation assembly according to the embodiments will substantially prevent the electrode(s) located on the lead from becoming dislodged or migrating from the implant location. In an embodiment, the lead fixation assembly has a low profile once installed and fully assembled so that, if the leads are intended to be chronically implanted, the cap can be located approximately 1.5 mm above the surface of the cranium. In addition, embodiments of the lead fixation assembly disclosed herein are comprised of only two separate components, namely, a cover and a cap, to simplify use of the assembly and fixation of the lead(s) in the assembly.

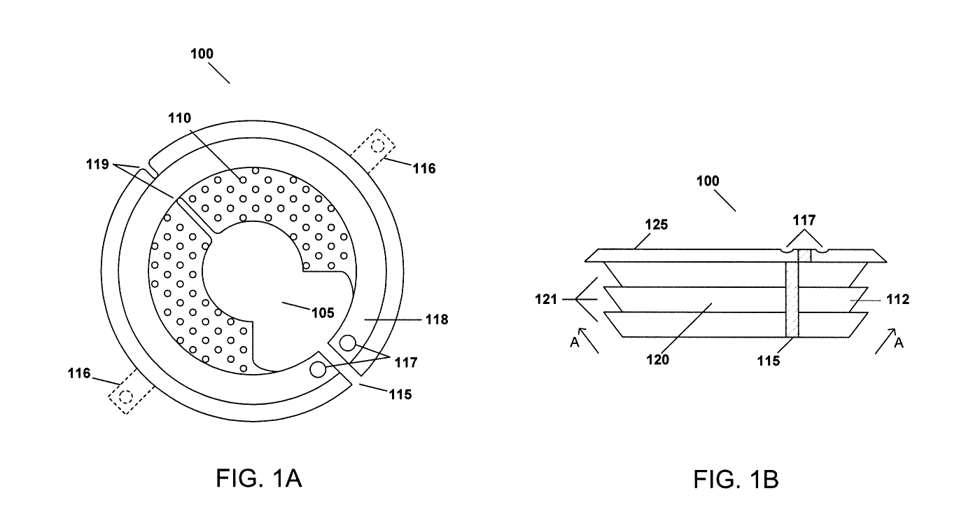

FIGS. 1A and 1B illustrate a top view and a side view, respectively, of a cover 100 of a lead fixation assembly according to an embodiment that is intended to be removably inserted into a burr hole formed in a subject. The cover 100 has a cover body 120 with a top surface 118 and a periphery 112. An aperture 105 is provided in the cover 100 to allow passage therethrough of one or more leads that have been implanted or that are to be implanted on or in the brain of a subject (interiorly of the cranium). That is, the aperture in the cover 100 can be threaded over the proximal portion of each lead or the body of the lead can pass through a gap 115 of the cover once the lead has been implanted. Alternatively, the cover 100 can be installed in the burr hole and then each lead can be introduced through the aperture 105 in the burr hole for implantation. The gap 115 and an optional live hinge 119 extend through the cover body 120 to allow the cover 100 to be inserted into and retained in the hole through which the lead(s) are passed. A plurality of raised surfaces 110 are provided in the top surface 118.

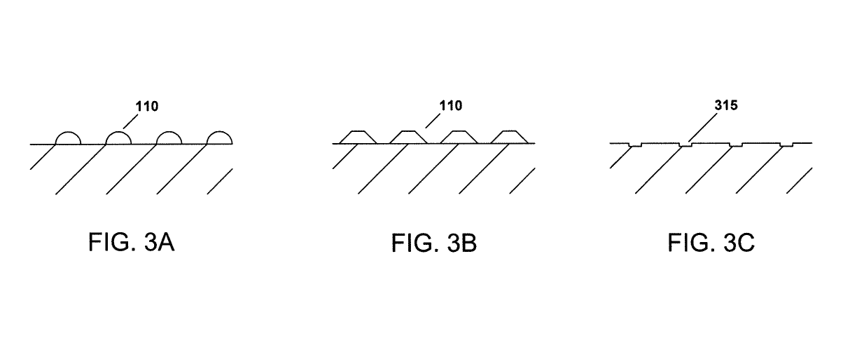

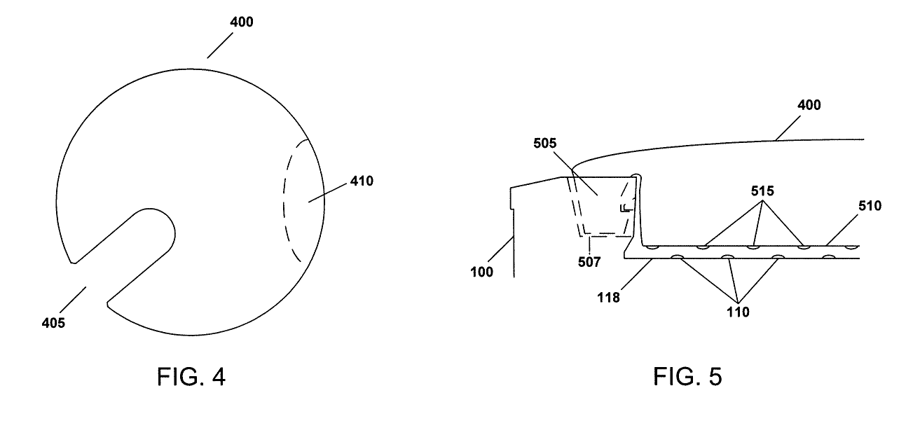

The cover 100, alone or together with a cap 400 (see, for example, FIGS. 4 and 5) is intended to permit a surgeon to secure a portion of the lead(s) passing through the aperture 105 relative to the exterior of the skull so as to discourage movement of the electrodes on the distal ends of the lead(s) away from the implant location. More specifically, the cover 100 is adapted to allow a surgeon to orient a portion of the implanted lead(s) over or among and between the raised surfaces 110 for a compression fit (or interference or friction fit) to limit post-implantation movement of the lead(s).

The cap 400 is provided for multiple purposes. For example, the compressive force that secures the lead to the cover either will compress the lead on the raised surfaces 110 (to the extent the lead is laid on top of the raised surfaces) or otherwise keep the lead in the intended position in the cover 100, as is discussed further below. The cap 400 also will minimize the exposure of interior of the cranium to the exterior of the cranium by covering the aperture 105. The cap 400 may also be provided with an opening or aperture 405 (e.g., a slot) through which the proximal end of each lead secured in the lead fixation assembly can exit the assembly and from there be routed to another device (e.g., a pulse generator or a measuring instrument). In an alternate embodiment, the opening through which the proximal end of the lead(s) can extend may be provided in the cover 100, such as in the cover body 120 or in the optional lip 125. In yet another embodiment, both the cap 400 and the cover 100 may be provided with features to allow the proximal ends of the lead(s) to pass between them (such as a partial slot in the cover and a mating partial slot in the cap). The cap 400 also presents a smooth surface to the soft tissue (scalp) reducing the risk of soft tissue erosion.

More particularly, and referring to FIG. 1A, the cover 100 is sized and shaped to fit within a defect or hole that has previously been formed or otherwise occurring in the cranium of a patient and through which one or more leads bearing one or more electrodes previously has been introduced or will be introduced into the brain tissue or to a surface of the brain. One typical size and shape for a burr hole is a circle of approximately 1.5 cm in diameter. The aperture 105 in the cover 100 may be provided with a number of possible sizes and shapes. The size and shape of the aperture 105 should be sufficient to allow the proximal ends of the implanted leads to be easily routed to whichever device the leads are to be connected (e.g., a neurostimulator implanted (or to be implanted) elsewhere in the cranium). In addition, if it is intended that the leads be implantable through the aperture 105 in the cover 100, the aperture should have dimensions sufficient to permit manipulation of the leads during the implantation procedure.

To use the cover 100, a portion of the lead extending away from the brain and the implant location and through the burr hole is situated so that it either lies on top of or between the raised surfaces 110. If this portion of the lead is oriented so that it rests on top of the raised surfaces, affixing the lead can be completed by fitting a cap 400 over the cover 100 as described below so that the lead is compressed between the cover and the cap and thus restrained from moving axially. If this portion of the lead is oriented so that it is routed between one or more of the raised surfaces 110, a friction or an interference fit alone may accomplish fixation, for example, if the raised surfaces are spaced closely enough together relative to the diameter of the lead, the lead will be compressed between the raised surfaces. In such an embodiment, subsequent addition of a cap 400 still may be desirable to provide added security for the fixation but not necessarily to provide the compressive force.

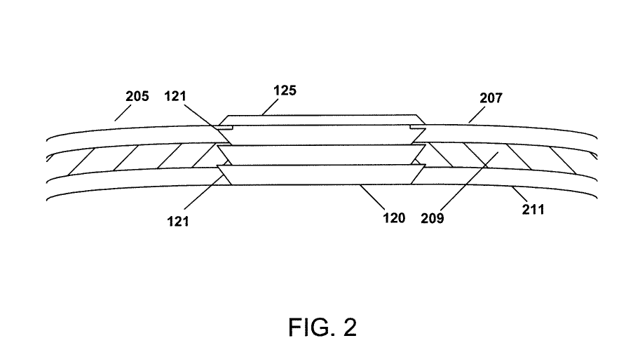

In alternative embodiments of the lead fixation assembly, the cover 100 may be provided with grooves 135 (as shown in FIG. 1D) or spaced-apart indentations or recesses 315 (as shown in FIG. 3C) in lieu of, or in addition to the raised surfaces 110. In these embodiments, portions of a lead to be fixed in the lead fixation assembly may be urged or pressed into one or more of the grooves or indentations for a friction fit.

In an embodiment, the dimensions of the raised surfaces 110 and the relative spacing of the raised surfaces can vary. In general, the raised surfaces 110 should be configured so that their shape will facilitate securing the lead, on the one hand, but not present any substantial risk of damaging it, on the other hand. The raised surfaces are substantially circular in FIG. 1A and substantially square in FIG. 1C. As further examples of possible shapes, raised surfaces 110 that are substantially hemi-ellipsoidal are shown in FIG. 3A and raised surfaces that are trapezoidal are shown in FIG. 3B. Many other configurations for the shape of the raised surfaces 110 will be apparent to those skilled in the art.

The spacing between the raised surfaces 110 in a given cover 100 desirably will be configured based on the intended use of the cover, that is, whether the lead will rest on top of the raised surfaces or will be routed in and among them, and, especially for the routed-in-and-among application, the diameter or range of diameters of the leads with which the lead fixation assembly will be used. A typical range of diameters for leads used in brain applications is 0.7 mm to about 2 mm. For example, the spacing between a given pair of raised surfaces 110 may be slightly less than the diameter of the lead in order to encourage an interference fit between the lead and the space between the raised surfaces. (The relative spacing between the raised surfaces may be more arbitrarily or imprecisely selected in relation to the diameter of the lead if, for example, the lead is to be laid on top of the raised surfaces 110 and then secured by interference fit with the cap 400, or the lead is to be routed in and among raised surfaces or among raised surfaces in a serpentine pattern or the like.) Alternatively, the diameter or width of any indentations 135 or grooves 315 may be provided to approximate or be slightly less than the diameter of the lead with which the assembly is designed to be used, to accomplish the interference fit during assembly.

Referring now to FIGS. 1A and 1B, the cover 100 may have one of a plurality of shapes, such as a circular perimeter or an elliptical, rectangular, X-shaped, or any other shaped periphery. In an embodiment, the cover 100 has a periphery that is designed to match the periphery of the hole that is formed (e.g., by a drill) in the patient's skull. Preferably, the cover 100 and cap (discussed below in connection with, for example, FIGS. 4-5) when fitted together, have a low profile such that the cover-and-cap combination does not appreciably extend outwardly from the skull after the assembly is positioned in a burr hole. In one embodiment, the cover-and-cap profile is approximately 1.5 mm after installation in a burr hole. In some embodiments, the cover 100 and/or the cap may be provided with a degree of curvature that approximates the curvature of the skull at the location at which the burr hole is formed, to minimize the overall profile of the assembly, for example, if the implant is intended to be chronic and the scalp is replaced over the burr hole and lead fixation assembly after the procedure.

When installed in the hole formed in the patient, the cover 100 is retained in place by a compressive force applied by the cover against the perimeter of the opening formed in the patient. Various features may be provided in the cover 100 to aid in securing it in the opening formed in the patient with a compression fit, either before or after the cover has been situated over the implanted lead(s). For example, in a substantially circularly-shaped cover 100 such as shown in FIG. 1A, a gap 115 may be provided creating two symmetric curved beams with a common support point. The beams can be deflected in a manner that allows the surgeon to reduce the diameter of the cover to facilitate its insertion by applying a force (e.g., by pinching the cover about the circumference). After the surgeon is satisfied with the position of the cover 100 in the hole, the applied force is released and the cover will attempt to return to its resting configuration, thus encouraging a friction fit between the cover and the interior surface of the burr hole. By way of another example, and referring still to FIG. 1A, a live hinge feature 119 may be provided at the common symmetric curved beam support point such that when the cover 100 is being compressed to install it in the opening formed in the patient, the compression exerted against the opening in the patient is more predictably managed.

The cover 100 further may be provided with one or more features to make it easier to manipulate the cover while it is being inserted into the burr hole. In FIGS. 1A and 1B, a pair of depressions 117 are provided in the top surface 118 on either side of the gap 115. The user can insert tweezers or a special purpose tool into the depressions 117 and use it to squeeze the cover 100 during insertion. Alternatively, protrusions such as pins (not shown) may be provided somewhere on the top surface 118 of the cover 100 to allow for gripping, with gloved fingers or a tool, to apply and release a force during insertion. In still another embodiment, a cover 100 may be provided with a combination of features (e.g., depressions and protrusions) to aid in securely inserting the cover into the burr hole.

If a cover 100 features any protrusions, such as pins oriented so that they are substantially perpendicular to the top surface of the cover, and a cap is used with the lead fixation assembly, the cap desirably may be provided with surfaces that are designed to receive, mate with or otherwise accommodate the protrusions so as to enhance the interconnection of the parts and/or keep the overall profile of the lead fixation assembly to a minimum.

In yet other embodiments, the top surface 118 of the cover 100 may be provided with other features designed to facilitate application of a compressive force for insertion, such as holes or slots that can be used to narrow or close the gap 115, with or without use of a tool.

The cover 100 may be provided with additional features in order to aid in retaining it in the desired position once it has been inserted into the burr hole. For example, to guard against the cover from being pushed all the way through the burr hole into the interior of the cranium, the cover 100 may be provided with tabs 116 (shown as broken lines in FIG. 1A) that extend horizontally from the periphery of the top surface 118 and that are adapted to receive bone screws or some other aid (not shown) for fastening the cover to the cranium. Referring now to FIG. 1B, a side view of an exemplary cover 100 for use in a lead fixation assembly is shown.

The cover body 120 may be provided with one or more features about its periphery 112 that are designed to enhance the fit of the cover in the burr hole and to further secure the cover in the hole. For example, the cover 100 may be provided with one or more protuberances 121 extending from the periphery 112 of the cover. The protuberances 121 are designed to displace or partially displace the cancellous bone exposed in the burr hole 209 or, alternatively, to be partially elastically deformed by the upper table 207 during insertion of the cover 100 and then to return or try to return to their resting formation once each protuberance 121 has passed through the upper table of cortical bone and impinges on the exposed cancellous bone 209. In an embodiment, the one or more protuberances 121 compact the cancellous bone 209 to further secure the cover 100 in the burr hole. The protuberances 121 can be directional as shown by the arrows A in FIG. 1B, so that they are generally oriented in a direction exterior of the skull. The protuberances 121 may comprise ridges that extend about substantially the entire periphery of the cover 100 or only about a portion of it. Alternatively, the protuberances 121 may be provided as discrete elements at one or more locations on the body of the cover 100.

In still another embodiment, the cover body 120 may be provided with threads (not shown) to help secure the cover 100 in the burr hole.

In another embodiment, the top surface 118 of the cover 100 may be provided as or with a lip 125 that is designed to discourage the cover from moving inwardly through the burr hole all the way into the cranial cavity during or after insertion. In an embodiment, the lip 125 is provided such that it extends over the outer edges of the burr hole upon insertion. Preferably, the lip 125 is formed integrally with the rest of the cover body 120 with the gap 115 extending uniformly therethrough. If the lip 125 is provided as a component that is separate from the cover body 120, the lip is provided with a gap corresponding to the gap 115 in the cover body. A lip 125 may also be provided with features to aid in retaining the lip and cover body 120 in the burr hole after the cover has been installed, such as tabs to accommodate bone screws and/or sutures.

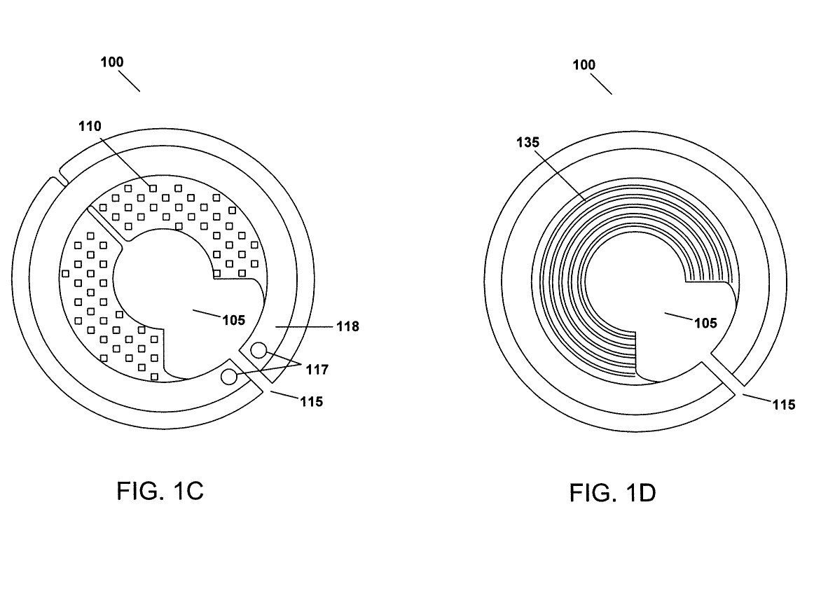

FIG. 2 is a representational view of an exemplary cover according to an embodiment after it has been inserted into a burr hole in a patient's cranium. As shown in FIG. 2, the cover 100 is located within a burr hole in the cranium 205, which comprises an outer cortical bone 207 (also referred to as, among other things, the outer table), a cancellous bone 209 (also sometimes referred to as spongy bone), and an inner cortical bone 211 (also referred to as, among other things, the inner table). In an embodiment, the one or more protuberances 121 of the cover 100 may press against the perimeter of the burr hole to discourage movement of the cover either into or out of the burr hole. The lip 125 of the cover 100 also may discourage movement of the cover into the burr hole.

FIG. 4 depicts an exemplary removable cap 400 for use in a lead fixation assembly according to an embodiment. As shown in FIG. 4, the cap 400 includes an opening or aperture 405. The opening or aperture 405 allows a portion of the lead proximal to the portion of the lead fixed in the cover 100 (i.e., a portion of the lead extending outwardly of the cover away from the body) to be passed through the cap 400 and routed to whatever the lead is intended to be connected with, such as an implanted pulse generator, EEG or ECoG recording instrumentation, etc. As discussed previously, in alternative embodiments, the exit point for the leads may be provided in the cover 100 rather than in the cap 400, or the exit point may be comprised of features in both the cover and the cap, such as partial slots that, when mated together, form an opening of sufficient size through which to pass the proximal end of each lead. Optionally, a recess 410 is provided to enhance the ease with which the surgeon can manipulate the cap 400 when fitting it over the cover 100 or removing it from the cover, whichever the case may be. The recess 410 may be designed of a size to accommodate a finger pad of the surgeon.

FIG. 5 is a representational view of a portion of an assembled cover 100 and cap 400 according to an embodiment. In this embodiment, the cover 100 and cap 400 are each provided with a feature to enhance the security of the fit between the cover and the cap of the lead fixation assembly. In the example shown, the cap 400 includes a flange 505 and the cover 100 is provided with a surface 507 to receive the flange when the cover and cap are mated. It will be apparent that any combination of locking features, for example, a flange on the cover and a receiving surface on the cap may also be provided, as long as the features will allow the proximal portion of the lead(s) to exit the lead fixation assembly after it has been assembled.

In an embodiment, an inward-facing surface 510 of the cap 400 (i.e., a cap surface that will face the top surface 118 of the cover 100 when the cap and cover are assembled together) may be provided with raised surfaces or indentations, such as 515, that are designed to mate with any raised surfaces 110, grooves 135, or indentations 315 that are provided in the cover. Although at least one lead would typically be arranged between the inward-facing surface 510 of the cap 400 and the top surface 118 of the cover 100 when locked together, such an arranged lead is not shown in FIG. 5.

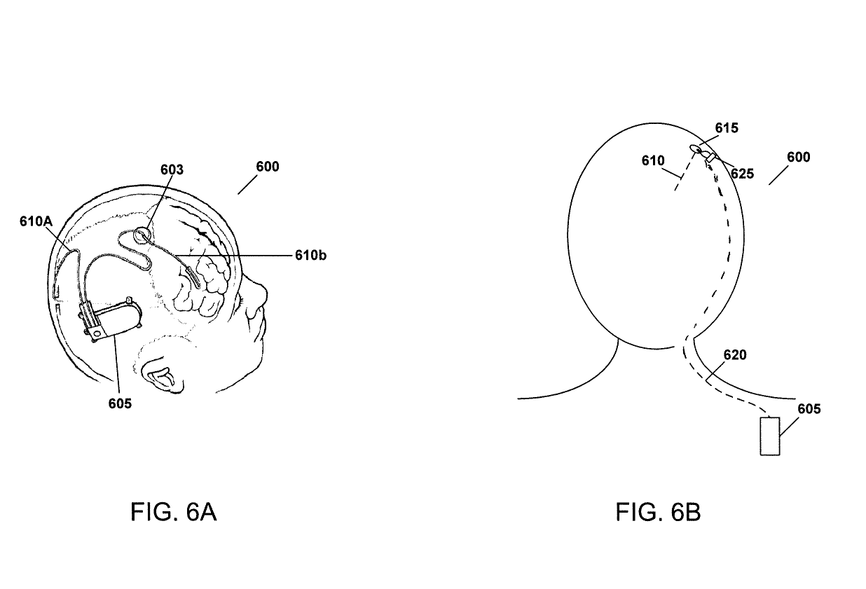

A method for using the lead fixation assembly according to embodiments in connection with two examples of a neurostimulation system or system for detecting neurological signals 600 is described with reference to FIGS. 6A and 6B. A burr hole 603 is formed in a patient's skull that extends through the entire outer table 207, the spongy or cancellous bone 209 sandwiched between the outer table and the inner table 211, and the inner table of the cranium.

One or more leads 610 are provided each having a proximal portion intended to remain exterior of the cranium and a distal portion intended to be implanted interior of the cranium of the patient. One or more electrodes (not shown) are provided on the distal portion of each lead 610. The electrode(s) may be configured to deliver an electrical signal to the brain (e.g., stimulate) or sense an electrical signal from the brain (e.g., detection or monitoring) or both. A given lead 610 may be provided with other features for communicating with the brain, including but not limited to features for delivering a drug to the brain, sensing chemical activity in the brain or delivering and sensing optical signals with respect to the brain. These features may be provided in a lead in addition to or in lieu of an electrode or electrodes.

Referring to FIGS. 6A, 1A and 2, installation of the cover 100 of the lead fixation assembly in a previously formed burr hole 603 will now be described. The surgeon compresses the cover 100 about its periphery 112 to close the gap 115 enough to allow the cover to be inserted into the burr hole 603 (i.e., into the defect created in the outer table, layer of cancellous bone, and inner table of the cranium).

If the cover 100 is provided with features to aid compression, such as depressions 117 or pins (not shown), then the surgeon can use these, perhaps in conjunction with tweezers or some other special tool that might be available, to fit the cover 100 into the burr hole 603. If a live hinge 119 is provided in the cover 100, it is expected that the ease with which the surgeon can install the cover will be enhanced, as the live hinge will provide a deflection point for the compressive force and will result in more predictable behavior while the surgeon is manipulating the cover in relation to the burr hole (the cover may include a circular component with a diameter on the order of approximately 15-20 millimeters in relation to the burr hole 603).

Referring now to FIGS. 1B and 2, if the cover 100 is provided with a feature to limit the degree to which the cover can be pushed into the burr hole 603, such as the lip 125, the surgeon can rely on this feature to guard against inadvertently pushing the cover all the way through the burr hole into the cranial cavity.

When the surgeon has the cover 100 in the desired position, the surgeon releases the compressive force and the cover, by virtue of the gap 115, will tend to expand about its periphery until it encounters the boundaries of the burr hole 603.

Referring again to FIGS. 1B and 2, if the cover 100 is provided with features designed to engage the interior walls of the burr hole 603, such as protuberances 121, these features will come into play as the compressive force applied to insert the cover is released. For example, if the cover body 120 is provided with protuberances 121 in the form of one or more ridges, the ridges will impinge against the spongy, cancellous bone 209 exposed by the burr hole 603 and will tend to deform the bone and help to secure the cover 100 in the burr hole. If any protuberances provided are further provided with directionality in the direction indicated by arrows A, the protuberances will tend to deform in the direction of the arrows A while the cover 100 is being installed, and then try to return to their resting state once installation of the cover is complete, further encouraging engagement of the cover body 120 with the walls of the burr hole 603.

Referring now to FIG. 6A, the distal end of a first lead 610A is routed through the burr hole 603 and the aperture 105 in the cover 100 and introduced into the brain tissue of the patient. The distal portion of the lead 610A is positioned so that the electrode(s) at the distal end of the lead are situated at an area that has been targeted for delivering therapy and/or measuring activity. Leads such as lead 610A are sometimes referred to as “deep brain” leads and the electrode(s) as “deep brain” electrodes or, when the electrodes are being used to stimulate brain tissue, “deep brain stimulation” or “DBS” electrodes.

Referring still to FIG. 6A, a second lead 610B having a strip or paddle-like portion at its distal end containing one or more electrodes 620 (this type of lead commonly being referred to as a “cortical strip” lead having “cortical strip” electrodes) is routed through the burr hole 603 and the aperture 105 in the cover 100. The distal end of the lead 610B, rather than being introduced into the brain tissue, is oriented so that it lies on a surface of the brain. (For example, when the system 600 is being used in an application involving treatment or detection of epileptic seizures, the cortical strip electrodes 620 may be positioned in an area that has previously been determined to be, or is suspected to be, the focus of epileptiform activity.) The number of leads 610 that are implanted and the order of implant will depend on the particular circumstances of each implantation procedure.

Leads 610, especially those intended for applications in the brain such as deep brain leads and cortical strip leads, typically are formed from a highly flexible materials (e.g., a conductive inner core (e.g., platinum) surrounded by an insulator and silicone). The flexibility allows the lead to be contorted without breaking it or any part of it, for example, when it is being connected to the device with which it is being used (e.g., an implantable neurostimulator). This flexibility, however, can make a lead 610 so pliant that it is difficult to control while it is being introduced into the patient. To improve control, a lead 610 is often provided with a lumen or inner core extending partially through its proximal end that receives a removable stylet to lend stiffness to the lead while it is being manipulated. After the lead is implanted, the stylet can be removed.

In an alternative embodiment, the cover 100 is installed in the burr hole after the lead(s) 610 have been implanted. In this method, the surgeon threads the proximal ends of the previously-implanted leads through the aperture 105 in the cover 100, taking care not to dislodge the distal ends of the leads from the locations at which they have been implanted. The leads also can be positioned in the aperture by orienting each lead body in the aperture via the gap 115. In still another alternatives, the leads are positioned in the aperture 105 in the cover 100 before the cover is installed and the leads are implanted, either by threading an end of each lead through the aperture, or by orienting the body of each lead in the aperture via the gap 115.

Once the cover 100 is installed and the lead(s) are implanted, a portion of each lead can be arranged on the top surface 118 of the cover. If the lead(s) have been provided with stylets that were used to provide stiffness during implantation, the stylets are removed before the arranging is accomplished. A portion of each lead that extends away from the brain and out of the aperture 105 is laid over the top surface 118. How the surgeon chooses to arrange the lead on the top surface 118 will depend on what features are provided in the top surface for lead fixation (e.g., raised surfaces 110, grooves 135, indentations 315, or some combination thereof). The arrangement further will depend on whether the cap 400 to be used with the cover 100 has any features designed to mate with any raised surfaces 110, grooves 135 or indentations 315 that are provided in the cover. The arrangement options may include (1) laying the lead over the raised surfaces 110, (2) routing the lead between and among the raised surfaces, (3) urging the lead into the grooves 135 or indentations 315, or (4) any combination of (1)-(3).

In an alternative embodiment, the lead(s) can be arranged, at least partially, on the top surface 118 of the cover 100 before the cover is installed or fully installed in the burr hole 603.

After each lead has been arranged on the top surface 118 of the cover 100, the surgeon further secures the arrangement by threading the cap 400 over the proximal end of each lead, and fitting the cap over the cover. The lead(s) will extend out of the aperture 405 in the cap 400, so that they can be connected to whatever equipment or device with which the leads are being used. If any features are provided to enhance the security of the fit between the cover 100 and the cap 400 such as the flange 505 and receiving surface 507 on the cover shown in FIG. 5, these features can be engaged at this time.

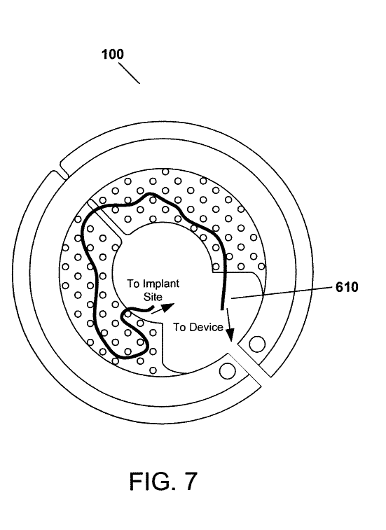

FIG. 7 depicts a top view of an exemplary cover with a lead according to an embodiment. As shown in FIG. 7, the cover 100 is configured to receive one or more leads, such as 610. Each lead 610 may be secured between one or more raised surfaces 110 (as shown in FIG. 7), indentations, and/or grooves of the cover 100. In addition, each lead 610 may be further secured when the cap is removably connected to the cover 100. Although only one lead 610 is shown in FIG. 7, more than one lead may be placed within the cover-cap assembly within the scope of this disclosure.

In an alternative embodiment, the cap 400 can be threaded over the proximal end of each lead at an earlier time, such as when a cover 100 is threaded over the proximal end of the each lead at the beginning of the lead fixation procedure.

If any features are provided to enhance the security of the fit of the cover 100 in the burr hole 603, these features can be engaged before or after the lead(s) are arranged on the top surface 118 of the cover or before or after the cap 400 is fitted onto the cover, depending on the surgeon's preference and the design of the components of the lead fixation assembly. For example, if tabs 116 are provided with screw holes for bone screws or furrows for sutures, the tabs can be used to fasten the cover 100 to the cranium.

Once the lead(s) have been affixed in the lead fixation assembly, the proximal end of each lead can be routed to the device or instrumentation with which the lead is intended to be used. Each lead may be provided with any of a fixed length or a predetermined range of lengths or so that it can be cut to any desired length at the proximal end.

A connector may be provided or available for each lead to facilitate connection for the given application, such as to connect the lead to a neurostimulator for delivering electrical stimulation through the electrode(s) or detecting signals generated by the neurons in the brain by the electrode(s) or both. FIG. 6A is a schematic representation of a cranially-mounted device 605 to which the proximal ends of deep brain lead 610A and cortical strip lead 610B are connected. FIG. 6B is a schematic representation of an extension lead 620 connected to a pectorally-implanted device 605. The pectorally-implanted device 605 in FIG. 6B may be used with a different extension lead 620 that is tunneled down from the vicinity of the burr hole through the patient's neck to the device. The extension lead 620 is connected to the lead(s) 610 implanted in or on the brain with an extender 625. It will be apparent to those skilled in the art that the lead fixation assembly can be used in numerous other applications, such as connecting the leads to external recording devices (e.g., not implanted).

If the lead fixation assembly subsequently needs to be removed, for example, because the leads need to be removed or relocated, both the cap 400 and cover 100 are removable. Any features that are provided in the cap 400 (e.g., recess 410 as shown in FIG. 4) or in the cover 100 (e.g., depressions 117 as shown in FIGS. 1A and 1B) can be relied upon, with or without aid of a tool, to remove the cap and cover from the burr hole.

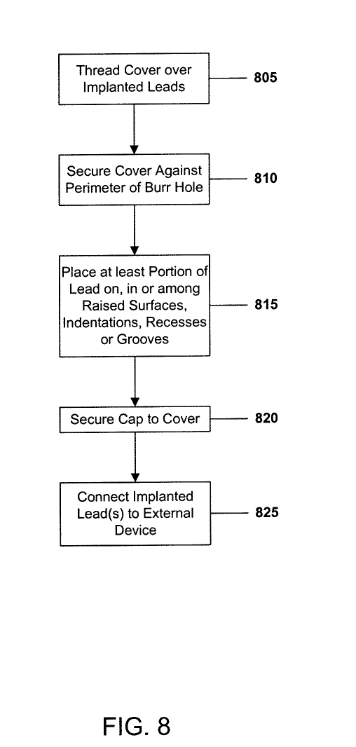

FIG. 8 depicts a flow diagram of an exemplary method of securing a lead in a burr hole cover assembly according to an embodiment. A distal end of one or more leads may be implanted on or within the patient's brain by feeding the leads through the burr hole. The one or more leads may be implanted at location(s) to which a treatment is to be provided and/or from which information is to be gathered.

A lead may include a stylet inserted in a lumen or a hollow core of the lead. The stylet may be inserted via the proximal end of the lead (i.e., the end of the lead that remains outside the patient) prior to implanting the lead. The stylet can be used to provide stiffness while the lead and the corresponding electrode(s) are guided to a desired location. The stylet is configured to provide rigidity to the lead, which is typically substantially non-rigid. The stylet may extend through the entire length of the lead or through only a portion of the lead. It may be provided with a handle that extends out of the proximal end of the lead to facilitate removal of the stylet after the lead has been implanted.

As shown in FIG. 8, a cover may be threaded 805 over the one or more implanted leads via the aperture in the cover. The cover is secured 810 against the perimeter of the burr hole using, for example, one or more of the techniques described above. In an embodiment, the cover is manipulated such that the cover is secured 810 within the burr hole. For example, the cover may be manipulated by using one or more features designed to enable the cover to be gripped, such as the depressions (e.g., depressions 117 in FIGS. 1A and 1B) and/or pins in the cover described above with which a gripping tool may be used. When the gripping tool releases the cover, the cover body 120 will try to return to its original non-deflected position, thus compressing the periphery 112 of the cover body against the perimeter of the opening formed in the body in which the cover is being installed, causing the cover to be secured 810 against the sides of the opening formed in the body. If a live hinge is provided, more control and/or predictability with regard to the deflection point and the force in the cover body 120 may be attainable. Alternately or additionally, the live hinge or the gap in the cover can cause the cover to press against the cancellous bone surrounding the opening formed in the body in order to secure 810 the cover therein. Alternately or additionally, a feature on the perimeter of the cover, such as tabs 116 in FIG. 1 or any other flexible protrusion, can engage the outer table of the cancellous bone causing the cover to be secured 810. In an embodiment, the cover may be secured 810 within the opening formed in the body prior to implanting the one or more leads.

A stylet, if provided in a lead, is withdrawn prior to fixation. Preferably, a stylet is withdrawn from a lead after the lead has been implanted on or within the patient's brain, but prior to placing 815 the lead on or in and among the one or more raised surfaces, indentations and/or grooves. As such, the lead has some rigidity during implantation and while the cover is being installed, but is flexible when the lead is placed 815 on or in and among the one or more raised surfaces, indentations, and/or grooves.

Upon placing 815 the one or more implanted leads on the top surface of the cover, the cap may be secured 820 to the cover. Securing 820 the cap and/or placing 815 a portion of a lead on or in and among the one or more raised surfaces, indentations, and/or grooves is used to secure the portion of the lead. For example, securing 820 the cap can compress and restrain the lead from moving axially relative to the cover-cap assembly. Similarly, placing 815 the lead in and among the plurality of raised surfaces, indentations, and/or grooves can restrain movement of the lead.

The proximal end of at least one implanted lead is connected 825 to an external device. In an embodiment, the external device may be one or more of a neurostimulator and a sensing device.

Although the lead fixation assembly has been described above with respect to a burr hole placed in the skull, the assembly may be used with respect to any hole that is made in or that otherwise occurs in bone or other tissue through which one or more leads are routed. In addition, the device to which the one or more leads are connected may be implanted on and/or within a patient's body. In other words, in addition to the embodiments described above, the stimulation source may be placed at a location external to a patient's body. Additional embodiments consistent with the teachings disclosed herein are included within the scope of this disclosure.

It will be appreciated that the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which are also intended to be encompassed by the following claims.

What is claimed is:

1. A lead fixation assembly, comprising:
a cover sized and shaped to be secured within a hole formed in the body of a patient, the cover having a first aperture, wherein a top surface of the cover comprises a plurality of elements configured to receive a portion of a lead; and
a cap having a second aperture designed to have a lead pass therethrough, wherein the cap is configured to be removably secured to the cover.

2. The lead fixation assembly of claim 1 wherein the plurality of elements in the top surface of the cover further comprises a first plurality of elements and a second plurality of elements configured to receive a portion of a lead, wherein each of the first and second plurality of elements has a shape, wherein the shape of at least one of the first plurality of elements is different from the shape of at least one of the second plurality of elements.

3. The lead fixation assembly of claim 1 wherein the cover further comprises a live hinge configured to assist in forming an interference fit with the hole formed in the body of the patient.

4. The lead fixation assembly of claim 1 wherein the cap further comprises a flange, wherein the cap is configured to be removably secured to the cover via the flange.

5. The lead fixation assembly of claim 1 wherein the cap further comprises an inward facing surface having at least one structure configured to receive the portion of the lead.

6. The lead fixation assembly of claim 1 wherein the cover further comprises one or more features configured to ease insertion of the cover into the hole.

7. The lead fixation assembly of claim 6 wherein the one or more features comprise one or more depressions or pins.

8. The lead fixation assembly of claim 1 wherein the cover further comprises a lip on the top surface, wherein the lip is configured to have a diameter greater than a diameter of the hole formed in the patient.

9. The lead fixation assembly of claim 1 wherein the cover comprises a lip on the top surface, wherein the lip is designed to prevent the cover from being inserted through the hole formed in the patient.

10. The lead fixation assembly of claim 1 wherein the cover further comprises one or more protuberances configured to assist in forming an interference fit with the hole formed in the patient.

11. The lead fixation assembly of claim 1 wherein the first plurality of elements comprises one or more of the following:
a raised surface;
an indentation; and
a groove.

12. A method of securing a lead in a lead fixation assembly, the lead having a distal portion intended to be implanted in a patient and a proximal portion intended to extend away from the implant site, the lead having at least one electrode at the distal portion thereof, the lead fixation assembly comprising a cover sized and shaped to be secured within a hole formed in a patient and having a first aperture and a plurality of elements adapted to receive the proximal portion of the lead, and a cap having a second aperture, wherein the cap is configured to be removably secured to the cover, the method comprising:
placing the proximal portion of the lead within the first aperture;
placing the proximal portion of the lead on or in and among the plurality of elements of the cover; and
removably securing the cap to the cover to affix the proximal portion of the lead between the cap and the plurality of elements such that the lead extending away from the implant site passes through the second aperture.

13. The method of claim 12 wherein removably securing the cap to the cover comprises removably securing the cap to the cover to affix the lead by an interference fit of the second portion of the lead with at least a portion of the plurality of elements of the cover and the cap.

14. The method of claim 12 wherein the lead further comprises a connector and further comprising:
connecting the connector to a stimulation source configured to provide stimulation to a portion of a body of a human patient via the electrode.

15. The method of claim 12, further comprising:
implanting the lead in the body of a human patient.

16. A lead fixation assembly, comprising:
a cover having a cover body having a periphery and a top surface, the cover adapted to fit within a defect or other opening formed or occurring in a body of a patient, the cover having an aperture extending through the cover body to allow passage therethrough of one or more leads, the top surface having a plurality of elements adapted to receive a proximal portion of each lead; and
a cap adapted to be juxtaposed with the cover, the cap having an opening configured to allow passage therethrough of one or more leads.

17. A method for using a lead fixation assembly, the method comprising:
orienting a portion of a lead implanted in a body of a human patient through an aperture in a cover having a cover body, a periphery, and a top surface characterized by a plurality of elements configured to receive a proximal portion of the lead;
securing the cover in a hole formed in a patient;
arranging the proximal portion of the lead on or in and among the plurality of elements; and
removably securing a cap to the cover, wherein the cap comprises an opening configured to pass the lead therethrough.

18. The method of claim 17, further comprising:
connecting a proximal end of the lead to an external device.

19. A method for using a lead fixation assembly, the method comprising:
securing a cover in a hole formed in the body of a patient, wherein the cover has an aperture, a cover body, a periphery and a top surface characterized by a plurality of elements adapted to receive a proximal portion of a lead implantable in the body of a patient;
threading a distal end of the lead through the aperture interiorly of the hole;
implanting the distal end of the lead in the body of the patient;
arranging the proximal portion of the lead on or in and among the plurality of elements; and
removably securing a cap to the cover, wherein the cap comprises an opening configured to pass the lead therethrough.

20. A method for affixing a lead implanted in the body of a patient in a hole formed in the patient, the method comprising:
orienting the lead in an aperture in a cover; and
arranging a proximal portion of the lead on or in and among a plurality of elements provided in a top surface of the cover to substantially prevent the proximal portion of the lead from moving relative to the cover.
